SEQUENCE LISTING (30 sequences)

SEQ ID NO: 1 - DNA, 18 bp, Artificial
  PCR primer R03C1_A
cggcaaacaatttttccg

SEQ ID NO: 2 - DNA, 18 bp, Artificial
  PCR primer R03C_C
acggaggtgttcacggag

SEQ ID NO: 3 - DNA, 18 bp, Artificial
  primer F59A3_A
cgtttgaaggatgatgtc

SEQ ID NO: 4 - DNA, 18 bp, Artificial
  primer F59A3_C
gatgctcgatgacttcgg

SEQ ID NO: 5 - DNA, 18 bp, Artificial
  primer C41D7_A
gattctcaagtccacccg

SEQ ID NO: 6 - DNA, 18 bp, Artificial
  primer C41D7_C
gacccgttctcctactcc

SEQ ID NO: 7 - DNA, 19 bp, Artificial
  primer M03F4_A
cgaaatggatctgagtggg

SEQ ID NO: 8 - DNA, 18 bp, Artificial
  primer
atatcccatgatgacccc

SEQ ID NO: 9 - DNA, 20 bp, Artificial
  primer C24A3_A
gagtgcgcttgaagagactg

SEQ ID NO: 10 - DNA, 20 bp, Artificial
  primer C234A3_C
cggaactcggagagagatag

SEQ ID NO: 11 - DNA, 20 bp, Artificial
  primer Y54G11A_A
ggatcttggctcctggaacg

SEQ ID NO: 12 - DNA, 20 bp, Artificial
  primer Y54G11A_C
cattgagtgatactcggccg

SEQ ID NO: 13 - DNA, 16 bp, Caenorhabditis elegans
tcgagaaatattcgaa

SEQ ID NO: 14 - DNA, 16 bp, Caenorhabditis elegans
attcgaaaaaacttcg

SEQ ID NO: 15 - DNA, 16 bp, Caenorhabditis elegans
attcgaaaaaacttcg

SEQ ID NO: 16 - DNA, 15 bp, Caenorhabditis elegans
ttccaaaaaaagaag

SEQ ID NO: 17 - DNA, 16 bp, Caenorhabditis elegans
aaagagtttttcgagg

SEQ ID NO: 18 - DNA, 16 bp, Caenorhabditis elegans
atttaacggactccaa

SEQ ID NO: 19 - DNA, 16 bp, Caenorhabditis elegans
acactgcggacaagtc

SEQ ID NO: 20 - DNA, 16 bp, Caenorhabditis elegans
tctagttggagtttat

SEQ ID NO: 21 - DNA, 16 bp, Caenorhabditis elegans
ttccctagtcttcggg

SEQ ID NO: 22 - DNA, 16 bp, Caenorhabditis elegans
ctttgtgatggcctgc

SEQ ID NO: 23 - DNA, 16 bp, Caenorhabditis elegans
aatataaagttcatgt

SEQ ID NO: 24 - DNA, 15 bp, Caenorhabditis elegans
cggagcagtagtgaa

SEQ ID NO: 25 - DNA, 16 bp, Caenorhabditis elegans
cgtcggatgtggcctt

SEQ ID NO: 26 - DNA, 26 bp, Caenorhabditis elegans
ggctctgaggtttcagaaaaatggct

SEQ ID NO: 27 - DNA, 17 bp, Caenorhabditis elegans
aaaagtaggcatcaccg

SEQ ID NO: 28 - DNA, 16 bp, Caenorhabditis elegans
aaagtacgcatcaccg

SEQ ID NO: 29 - DNA, 16 bp, Caenorhabditis elegans
gatcatttttgcccga

SEQ ID NO: 30 - DNA, 16 bp, Caenorhabditis elegans
gatcactttttcccga

(19) United States
(12) Patent Application Publication
(10) Pub. No.: US 2004/0161782 A1
(43) Pub. Date: Aug. 19, 2004

(54) Means and methods for identifying genes and proteins involved in the prevention and/or repair of a replication error

(76) Inventors: Marcel Tijsterman, Oegstgeest (NL); Ronald Hans Anton Plasterk, Bussum (NL)

Correspondence Address: TRASK BRITT, P.O. BOX 2550, SALT LAKE CITY, UT, 84110, US

(21) Appl. No.: 10/719,995

(22) Filed: Nov. 21, 2003

Related U.S. Application Data
(63) Continuation of application No. PCT/NL02/00322, filed on May 22, 2002.

(30) Foreign Application Priority Data
Nov. 28, 2002 (GB) WO 02/095071
May 22, 2001 (EP) 01201936.0

(51) Int. Cl. C12Q 1/68

(52) U.S. Cl. 435/6

(57) ABSTRACT

The invention enables a person skilled in the art to determine whether a product of a gene is involved in the prevention of a mutation. Identified genes can be used to develop diagnostic tools or used as a target for drug development to manipulate cells on the basis of the presence or absence of function of this gene. Since DNA instability is one of the reasons for rapid tumor progression, gene therapy with products of such genes may be used to treat cancer.

### CROSS-REFERENCE TO RELATED APPLICATIONS

This application is a continuation of International Application No. PCT/NL02/00322, filed on 22 May 2002, which was published in English on Nov. 28, 2002, as International Publication No. WO 02/095071, designating the United States of America, the entire contents of which are incorporated by this reference.

### BACKGROUND

Technical Field: The invention relates to the fields of molecular biology and medicine, more particularly, the invention relates to the identification and use of cellular pathways that are important for maintaining DNA integrity in a cell.

Human tumors arise by multiple mutations that turn so-called proto-oncogenes into active oncogenes, and/or inactivate tumor suppressor genes. Each of these events is the result of a somatic mutation. The chances of getting the “right” combination of mutations to turn a normal cell into a tumor cell are very small, given the inherent stability of the genome. These chances are, of course, much enhanced if one of the earliest events in the genetic pathway from normal cell to tumor cell is a mutation that enhances the overall level of mutations. Such mutations are called “mutator” mutations.

As a simplified calculation to illustrate this: say that six mutations are needed within one clonal cell line. Assume that in a mutator cell line the level of mutations is 100 times higher than in a wild-type cell. Then the chance of the combination of six mutations that make a full-blown cancer cell is 100 to the 6th power higher than in a non-mutator cell, or 10 to the 12th power. Such calculations are quite old, and in a sense it could not have been a surprise when it was found that indeed many human cancer cells are mutators.

One common type of mutator genes is DNA mismatch repair. This system recognizes small DNA replication errors, and corrects them. The replication machinery tends to slip on stretches or simple repeat sequences; the resulting repeat instability is also prevented by DNA mismatch repair. Many human tumors are apparently defective in mismatch repair; since one can recognize repeat instability. Indeed, in approximately 50% of these tumors one can find a mutation in the known DNA mismatch repair genes (such as MSH and MLH genes). This confirms that indeed an early event in tumorigenesis is a chance mutation that damages a system that serves to stabilize the genome; then in the resulting unstable genetic background it is much more likely than before that the oncogenic mutations can occur.

Mismatch repair genes were not originally discovered in tumor cells. The known DNA mismatch repair genes were initially discovered in unicellular model organisms (bacteria), as mutator mutants, in which the levels of DNA mutations were enhanced. One case of a hereditary human cancer (HNPCC) was found to be caused by a mutation in a mismatch repair gene, and subsequently one could inspect all the known homologues of factors involved in bacterial mismatch repair for a role in human cancers. But how to get to the other mutator genes? It is known that in some classes of tumors 50% of the tumors that show repeat instability do not show a mutation in a known mismatch repair gene, and must thus harbor a mutation in another mutator gene. On top of that, there may be mutators that affect mutation levels without showing repeat instability, and thus the actual number of human cancer-causing mutators may be higher than now known.

How to get to these genes? Again, model organisms must help to indicate candidate genes. Homologues of these genes may then be inspected in human tumor samples for possible inactivating mutations. Such candidates, if selected from non-human sources, ideally fulfill the following criteria:

1. Loss or reduction of function of the gene must result in a significantly enhanced mutation rate in the cell lineage.

2. There are homologues in the human genome.

Since animals are in many respects different from bacteria, it is possible that some genome stabilizing systems are not present in bacteria, but are unique to animals. Therefore, these mutator genes are ideally sought in an animal system. On the other hand, many factors involved in DNA metabolism, cell cycle, etc. are very conserved in evolution, so that one may be able to discover relevant genes in simple non-vertebrate model animals.

DNA mismatch repair (MMR) mutants were originally found in screens directed at the identification of bacterial mutants that had a mutator phenotype, and thus had elevated levels of spontaneous mutations in their progeny. Subsequent genetic, as well as biochemical, studies identified the mismatch repair machinery as an enzymatic complex that could recognize DNA mismatches resulting from single nucleotide substitutions or small insertions/deletions, that could recognize the parental from the newly synthesized strand, excise the new strand around the lesion, and initiate repair to close the gap.

One of the greatest success stories of model organism genetics came when a human syndrome of cancer predisposition, HNPCC for Human Non-Polyposis Colon Cancer, was found to result from a defect in human homologues of genes encoding components of the bacterial mismatch repair machinery (Fishel et al., 1993; Leach et al., 1993; Bronner et al., 1994; Kolodner et al., 1994, 1995; Liu et al., 1994; Nicolaides et al., 1994; Papadopoulos et al., 1994). The fact that cancers are typically characterized by an increased instability of simple DNA repeats provided the first clue that a replication-associated repair mechanism was involved (Peinado et al., 1992; Aaltonen et al., 1993; Ionov et al., 1993; Peltomaki et al., 1993). The notion that MMR defects are associated with human cancer provides strong support for the hypothesis that a so-called mutator phenotype, here as a result of elevated levels of unrepaired somatic DNA mismatches, can promote tumorigenesis (Loeb, 1991). This model has been further supported by mouse knockouts of the MMR genes msh-2, msh-6, Pms-2 or Mlh-1 that show enhanced cancer frequencies and repeat instability (de Wind et al., 1995; Reitmar et al., 1995; Edelmann et al., 1996; Baker et al., 1996; Narayanan et al., 1997; Prolla et al., 1998).

Also in humans that do not contain germline mutations in DNA mismatch repair genes, tumors are often found to display repeat instabilities. Upon analysis, these tumors are sometimes defective in known components of the MMR machinery; either they carry mutations within the genes themselves or the expression of these MMR genes is epigenetically down-regulated as a result of hypennethylation (Kane et al., 1997; Cunningham et al., 1998; Herman et al., 1998; Veigl et al., 1998). Interestingly, not all sporadic human tumors with repeat instability show a defect in the known DNA mismatch repair genes (Liu et al., 1996). In addition, in approximately 30% of HNPCC cases no germline mutations were found in the known MMR genes (Peltomaki and de la Chapelle, 1997; Lynch and Smyrk, 1998). This suggests that there are additional genes in humans and also in other organisms or cells, whose loss results in this specific type of genetic instability. These genes cannot be easily traced; the currently known genes were only traced based upon prior insights into the mechanism of DNA mismatch repair in model organisms.

### BRIEF SUMMARY OF THE INVENTION

In one aspect, the invention provides a method for determining whether a product of a gene is involved in preventing a replication error in a cell comprising providing the cell with a specific inhibitor of the product and determining the level of functional expression of a marker gene in the cell, wherein the level of expression of the marker gene is dependent on the occurrence of a replication error. With this method, it is not only possible to determine whether a gene is directly involved in preventing a replication error, it is also possible to determine whether a gene influences the efficiency with which the process occurs.

Replication errors usually comprise nucleic acid deletions, nucleic acid insertions and/or base alterations. Replication errors typically occur when mismatch repair systems fail to correct mutations that occurred between two division cycles. Replication errors can affect the level of functional expression of a marker gene in many different ways. For instance, modification of an enhancer or silencer sequence involved in regulating expression of the marker gene. A replication error can also lead to a change in the coding region of the marker gene whereby the change results in a reduction or complete abolishment of the activity of a gene product of the marker gene. Another example of the expression level of a marker gene being dependent on a replication error is the disappearance or appearance of an altered epitope in a gene product of the marker gene as a result of the replication error, the epitope being detectable with a binding molecule specific for the epitope. Thus, many different types of replication errors can influence functional expression of the marker gene.

In a preferred embodiment of the invention, the replication error comprises nucleic acid repeat instability. Nucleic acid repeat instability is a form of replication error that occurs particularly frequently. Several genes have been shown to be involved in preventing nucleic acid repeat instability in a cell. Typical examples are msh-2, msh-6, Pms-2 and Mlh-1. An absence of expression of these genes has been correlated with enhanced cancer frequencies. Using the method of the invention, it is possible to find additional genes involved in preventing a replication error in a cell. The invention is particularly advantageous for finding additional genes involved in preventing nucleic acid repeat instability in a cell.

### BRIEF DESCRIPTION OF THE DRAWINGS

FIG. 1. The C. elegans msh-6 gene. (A) Structure of the C. elegans msh-6 gene deduced from genomic sequences and cDNA generated by RT-PCR from Bristol N2 RNA. The genomic region that is deleted in pk2504 (nt. 24180-25956 of Y47G6A, GenBank accession number AC024791), and takes out exon-5 and part of exon-6, is indicated. (B) Alignment of the amino acid sequence of C. elegans, Human and S. cerevisae MSH-6 using the CLUSTALW algorithm. Black shading indicates amino acid identity, grey shading indicates conserved amino acid substitutions. The amino acids deleted in pk2504 are underlined. Possible alternative splicing of exon-4 on to exon-7 predicts an out-of-frame product.

FIG. 2. Mismatch repair proteins MSH-6 and MSH-2 protect the C. elegans germline from spontaneous mutagenesis. The experimental setup that is used to measure the level of spontaneous mutagenesis is described in the materials and method section. This assay determines the absolute number of loss of function mutations in essential genes in a region that covers approximately 7% of the C. elegans genome (estimated number of target genes: ˜300). The y-axis reflects the percentage of animals that acquire such a lethal mutation within one generation.

FIG. 3. Outline of the principle to detect somatic repeat instability.

FIG. 4. Genetic instability in MMR-defective somatic cells. A schematic representation of the constructs that are used to measure somatic repeat instability is depicted above the images of the nematodes. (A) Transgenic C. elegans that carry multiple “in-frame” copies of heat-shock driven LacZ. (B) MMR-proficient transgenic C. elegans (N2) that carry multiple copies of a LacZ-containing construct in which a repeat sequence is cloned immediately downstream of the ATG that puts the downstream positioned β-galactosidase ORF out-of-frame. (C) The identical transgenic array crossed into an msh-6 genetic background.

FIG. 5. C. elegans populations fed on E. coli that produce dsRNA homologues to the C. elegans genes unc-22 (A) and msh-6 (B).

FIG. 6. Schematic representation of the high throughput RNAi-based screens to identify novel mutator loci: Individual animals are fed on dsRNA-producing bacteria, the progeny are collected and assayed for beta-galactosidase activity.

### DETAILED DESCRIPTION OF THE INVENTION

As used herein, the term “replication error” means not only errors that occur during replication, but errors occurring before replication. Such errors can become fixed in the genome, upon replication of the DNA. The term “replication errors,” therefore, refers to errors that are introduced into the DNA and that are stable, or stabilized during replication of the cell.

Preventing a replication error in a cell may be done in many ways. Typically, preventing a replication error is achieved by preventing fixation of a mutation in the genome by means of replication of the cell. This can, for instance, be achieved by improved repair of mutations such that typically more mutations are corrected prior to fixation. Another method for preventing a mutation error from becoming fixed in the genome is to (temporarily) inhibit cell division, thus allowing more time in which the mutation can be repaired by the repair machinery of the cell.

For the present invention, the phrase “functional expression of a marker gene” means expression of a detectable part of a product of the marker gene. Preferably, activity of a product of the marker gene is detected. However, detection of functional expression can also be done by means of detecting the presence of a particular epitope specific for a gene product of the marker gene. Activity of a promoter or even total amount of a marker gene product, protein, may stay essentially the same, as long as, only one epitope of a product of the marker gene is altered or introduced upon the replication error.

Any method for specifically inhibiting a product of a gene in a cell is suitable for performing the invention. However, a particularly suitable gene-specific inhibitor comprises gene-specific RNA. Anti-sense RNA, for instance, is very effective in significantly reducing expression of specific genes, particularly in plants cells. Anti-sense RNA can also be very effective in animal cells. In a preferred embodiment, the specific inhibitor comprises gene-specific double-stranded RNA. Specific double-stranded RNA and particularly RNAi (Fire et al., 1998, Fraser et al., 2000) is very effective in significantly reducing expression of specific genes, also in mammalian cells (Brummelkamp et al., 2002; Elbashir, 2001). In a particularly preferred embodiment, the specific inhibitor of a gene product comprises RNAi. A gene-specific inhibitor does not necessarily have to be specific for only one gene. A gene-specific inhibitor can also be specific for a collection of genes as long as the collection of genes comprises a region of significant homology.

It is possible to use any type of cell in a method of the invention. Culture cells are particularly accessible for manipulation. Moreover, these types of cells can be grown to large numbers, thus facilitating detection of expression of marker genes. However, cell culture cells have a drawback in that many of them already contain unstable genomes. Therefore, it is preferable to study genome stability in the context of a complete animal. In a preferred embodiment, the organism comprises C. elegans. C. elegans contains a limited number of cells of which the differentiation route and ancestry are completely resolved. In a preferred embodiment, the non-human organism is transgenic for the marker gene. In this case, it is possible to identify cell type-specific genes involved in preventing a replication error in a cell. The method allows one to screen all genes in the C. elegans genome systematically for their possible role in maintaining chromosome stability. A transgenic animal was constructed in which a calorimetrically visualizable gene (lacZ) would only be expressed after a mutation in a short DNA repeat sequence. It was confirmed that, indeed, in such a transgenic animal, one could see little patches of blue cells, but only if one had inactivated a known DNA mismatch repair gene (such as MSH, mentioned above). It was then found that the same effect can be reached if the MSH gene is inactivated, not by mutation but silenced by a phenomenon called RNA interference (RNAi). An advantage of RNAi is that it does not completely knock out gene function in all cells of the body, so that RNAi effects can be detected even if the silenced gene is itself essential for life; in that case, RNAi on a population of animals will perhaps result in many early deaths, but in the few escaping animals, it was found that blue patches can still be scored that result from the mutator effect (Tijsterman et al., 2002).

Using this method, all 2000 genes were initially studied that map on chromosome I of C. elegans. Among the genes found to have a mutator effect are plausible candidates, such as the cell cycle checkpoint genes cdc-1 and cdc-5, and the rpa-2 gene, is a homologue of gene known to be involved in DNA repair in yeast. In addition, there are also genes whose function was thus far unknown (see table 3). This analysis was extended to approximately all 19000 genes encoded by the C. elegans genome. Genes found to have a mutator effect are listed in table 4.

A method is described that allows one to detect genes that are likely candidates to be the cause of a high proportion of human tumors. Such genes are useful in diagnosis and treatment choice. Tumors of one mutator type may have a different prognosis or response to a given therapy than another. This can be tested once these mutator genes are known (along with their human counterparts). Such genes are also useful in the design of new drugs. Of course, tumors are only detected once the genetic damage has been done, but still the chances of additional new instability (for example, leading to an escape from drug chemotherapy by mutations in drug-resistant genes) will go down upon the chemical activation of parallel mutator pathways, or by gene therapy based repair or by strengthening the damaged mutator gene's function. Knowledge of the common mechanisms that cause human cancers aids in defining strategies that protect individuals against such mutator effects, and is thus a form of prevention. Other uses entail life style or dietary advice, food supplements, etc. The invention, therefore, also provides the use of a mammalian, and preferably human, homologue of a gene obtainable by a method of the invention in a method for diagnosis, prognosis, gene therapy and drug targeting approaches.

Any gene can be a marker gene provided that a product of the gene can be detected. Expression of the marker gene and particularly changes in the expression level of the marker gene, must be detectable. Preferably, the marker gene is not performing a critical function in the cell. Preferably, the marker gene is provided to the cell. Suitable marker genes are LacZ and GFP, although other equally suited marker genes are readily available. In a preferred embodiment, the marker gene comprises LacZ.

Many types of replication errors can result in a change in the level of expression of a marker gene in a cell. In a preferred embodiment, the replication error comprises an error that results in a frame-shift in a protein-coding domain of the marker gene. In a particularly preferred embodiment, the replication error comprises a deletion/insertion in or of a mono- or di-nucleotide repeat and wherein the deletion and/or insertion results in a frame-shift in or of the protein-coding domain, wherein the frame-shift results in a change in the level of functional expression of the marker gene. In a preferred embodiment, the frame-shift results in a functional protein, preferably an easily detectable function that is not critical to the cell. Detection of the function can subsequently be used to measure the level of functional expression of the marker gene. Preferably, the frame-shift results in functional LacZ or GFP expression.

In one aspect, a method of the invention further comprises identifying the gene involved in preventing nucleic acid repeat instability in a cell. Once identified, a person of ordinary skill in the art may isolate the gene through methods known in the art. It is also possible to synthetically generate the gene. The invention also provides an isolated and/or recombinant gene obtainable by a method according to the invention. In a preferred embodiment, the isolated and/or recombinant gene comprises a sequence as listed in table 3 or table 4 or an equivalent thereof. An equivalent of a gene as listed in table 3 or table 4 is preferably a human homologue thereof.

A significant fraction of human tumors are apparently caused by somatic mutations in genes that affect genome stability, but these mutations are not always in genes of the known mismatch repair system. Previously, there was no direct way to identify these genes, while they may be highly relevant as causative agents of human cancers. An aspect of the present invention provides a system that mimics the somatic repeat stability in human cancers. With the means and methods of the invention, it is possible to determine whether a product of a gene is involved in preventing a replication error in a cell. It is further possible to identify the product and the gene. Identified genes can be isolated and/or cloned. Such isolated and/or recombinant genes can further be used in a large variety of methods known to the person skilled in the art. In a preferred embodiment, the invention provides a method for determining whether a cell is predisposed to display a nucleic acid repeat instability phenotype comprising determining functional expression of a gene according to the invention in the cell or derivative thereof. Preferably, the gene is a gene as listed in table 3 or table 4 or an equivalent thereof. Preferably, the equivalent is a human homologue of a gene listed in table 3 or table 4. Human homologues may be found by sequence comparison. Human homologues may also be found based on a function of the proteins in the two species. A homologue of a gene identified in a method of the present invention, comprises a similar function in another species, not necessarily a similar amount, as the gene identified with a method of the invention. A nucleic acid repeat instability phenotype is, for example, cancer or an immune deficiency. The method may be performed through any means for determining whether a gene is expressed in a functional way. One way is to determine whether the gene is intact in the cell. Typically, this is done on a nucleic acid sequence level. Alternatively, expression levels can be detected by means of, for example, an antibody specific for a proteinaceous product of the gene in the cell or a method for detection of RNA. In a preferred embodiment, the cell is present in a clinical sample. In this way, it may be determined whether an individual is predisposed to developing a disease associated with instability of the genome. The method may be used advantageously to determine whether an individual is predisposed to display a nucleic acid repeat instability phenotype. In addition, diagnostic tools of the invention may also be used, alone or in combination with other methods, to determine whether the cell is a cancer cell or predisposed to become a cancer cell and which type of mutator mutation is responsible for its etiology (which may play a role in prognosis, therapy choice and possibly in therapy development). The cell may, of course, also be part of, or be derived from, a non-human organism. In this way, individuals may be found, or screened for, that have alterations in the functional expression of the gene.

The invention further provides a kit for performing a method for determining whether a cell is predisposed to display a nucleic acid repeat instability phenotype, the kit comprising a means for determining functional expression of a gene identifiable with a method of the invention. Preferably, the kit comprises an antibody specific for a gene product of a gene identifiable with a method according to the invention. In a preferred embodiment, the kit comprises a probe for a gene identifiable with a method of the invention or a probe for a gene product of the gene. In yet another aspect, the kit comprises means for obtaining at least a functional part of a sequence of a gene identifiable with a method according to invention, or a functional part of a sequence of a gene product of the gene. A functional part of a sequence comprises at least a part sufficient for the identification of the gene (gene product) and/or the determination of whether the gene and/or product derived from it comprises an alteration such that its activity in preventing a replication error in a cell is modified and preferably decreased. Typically, a functional part comprises at least 20 nucleotides or 7 amino acids.

The invention provides means and methods for identifying genes and gene products involved in preventing a replication error in a cell. With the tools provided by the invention, it is possible to identify essentially all genes and/or gene products involved in the prevention of a replication error in a cell. The identification aspect of the invention is exemplified below for C. elegans. Of course, this is just one way of obtaining the desired result. Most research on mismatch repair function in vivo has focused either on unicellular organisms such as bacteria or yeast, because in those organisms one can easily monitor mutator effects in large numbers of progeny, or in somatic cells or tissue culture cells of higher animals. The numbers of progeny animals that need to be inspected to recognize spontaneous mutants (that are not induced by chemicals or radiation) is prohibitively large. It was, therefore, possible, but not established, that the mismatch repair machinery contributed significantly to removal of point mutations from progeny in multicellular organisms. It is possible that the mismatch repair system acted only to protect against base pair substitutions that arise in somatic cells. However, it was found that the mismatch repair system in a metazoan animal, such as C. elegans, has pretty much the same effect on progeny that it has on that of unicellular organisms: a 20× decrease in the mutation rate with most mutations being transitions and frame-shifts.

In C. elegans, this protection is as important for the male germline as for the female (actually hermaphrodite) oocytes. Note that the role of DNA mismatch repair in hermaphrodite sperm was not addressed, since experimentally, the mutations that arise in self-fertilizing hermaphrodites cannot easily be attributed to the sperm or the oocytes.

Genes capable of preventing a replication error in a C. elegans cell may be used to screen for homologues of the gene in other organisms. It is likely that such a homologue will also have the property of preventing a replication error in a cell of that organism. A person skilled in the art is well capable of verifying this property in a homologue. Particularly preferred homologues are, of course, human homologues.

The level of spontaneous mutagenesis in the msh-6 mutant strain per generation is 10-fold lower than that induced by the most efficient chemical mutagens. Therefore, it is not surprising that one recognizes different visible mutants among progeny of msh-6 animals. Since the mutator effect is continuous, one could, in principle, culture the strain for multiple generations and achieve quite significant accumulated levels of mutations (while maintaining selection pressure for viability). A strain like this may be of use in experiments aimed at experimental quantitative genetics, where genetic adaptation to specific environmental challenges can be studied more efficiently that in a wild-type isolate, because the rate of evolution is enhanced.

One of the most spectacular aspects of RNA interference is that it also works when C. elegans is fed on dsRNA or even on E. coli strains that are genetically modified to produce C. elegans-specific dsRNAs (Timmons and Fire 1999). Thus far, these effects were always transient and did not persist longer than two or three generations, when apparently the RNAi machinery had been diluted out. Since a gene whose function is to protect the genome against mutations was studied herein, it was found that a single episode of exposure to dsRNA was sufficient to induce permanent mutations in the progeny of exposed animals. Fortunately, for higher animals than these small worms, there is no evidence that ingested nucleic acids can affect the germline. Since the effect can also be induced by feeding dsRNA for the mismatch repair genes, a system to test any C. elegans gene for its role in repressing repeat length changes is obtained. Recently, genome-wide libraries of dsRNAs of C. elegans have been described (Fraser et al., 2000), and testing all genes in this animal's genome for their mutator effect is now being done. Additional classes of mutator genes may exist, possibly, not at all related to mismatch repair, but perhaps to replication factors, chromatin proteins that protect the genome, or totally novel protection systems, they can now be discovered, and human homologues may be tested for their role in human cancer etiology.

The invention provides means and methods for determining whether a cell is disposed to display a replication error, making it possible to devise means and methods that capitalize on this capability. In one aspect, the invention provides a method for determining whether a compound is capable of influencing a process involved in preventing a replication error in a cell comprising providing the cell with the compound and determining the level of expression of a marker gene in the cell, wherein the level of expression of the marker gene is dependent on the replication error. Preferably, the level is dependent on the occurrence of the replication error. In a preferred embodiment, the compound is provided to a collection of the cells. A compound is said to influence the process when the compound reduces or increases the frequency with which a replication error is detected. In a preferred embodiment, the method further comprises providing the cell with a specific inhibitor for the expression of a gene involved in preventing a replication error in a cell. In this way, the detection of compounds capable of decreasing the frequency is enhanced. Preferably, the gene is a gene obtainable by a method of the invention.

In yet another aspect, the invention provides a gene delivery vehicle comprising a gene of the invention or a functional part, derivative and/or analogue thereof. Such a functional part, derivative and/or analogue comprises the same type of nucleic acid repeat instability preventing activity as the gene, but not necessarily the same amount of activity. The invention further provides a method for influencing a process involved in preventing a replication error in a cell comprising providing the cell with a gene delivery vehicle of the invention. In this way, the cell can be provided with an improved capacity to prevent nucleic acid repeat instability. In one aspect, the invention therefore provides the use of a gene delivery vehicle of the invention for the preparation of a medicament.

As used herein, the term “gene” may refer to a protein-coding domain, which may or may not be accompanied by or with local cis acting regulatory elements. Typically, cis acting regulatory elements are promoters, transcription terminator elements, introns and the like. A gene product may be a transcribed RNA and/or a translated proteinaceous molecule. With the current technology, synthetic versions of each of such RNA or proteinaceous molecule may be generated. Such synthetic versions are, of course, equivalents.

In yet another aspect, the invention provides a non-human animal comprising a marker gene wherein the expression level of the marker gene is dependent on the occurrence of the replication error. Such an animal can be favorably used in a method of the invention. Preferably, the marker gene is provided to cells of the animal. In a particular preferred embodiment, the animal is transgenic for the marker gene. The invention also provides a method for determining whether a compound is capable of inducing a replication error comprising providing a non-human animal according to the invention, with the compound determining in the animal or progeny thereof whether the expression level of the marker gene is altered. Preferably, the non-human animal comprises C. elegans.

The compound can be any compound. In case where the compound comprises RNAi, it is possible to study whether the RNAi is capable of inducing a replication error. When the RNAi is designed to be a specific inhibitor for a gene product of the animal, then the method resembles methods that are described herein. When no specific designing is done, then it is still possible to study the capability of the RNAi to induce a replication error. Thus, RNAi, which may be designed to inhibit a specific gene product or a library of sequences, may be used to study the capability of the RNAi to induce a replication error.

In another embodiment, the compound comprises a free radical or a substance capable of generating a free radical, either alone or in combination with another molecule. In general, this method is suited to determine and identify compounds that are capable of inducing replication errors in whole organisms.

The invention further provides a method for typing a cell comprising determining in a sample the cell functional expression of a gene listed in table 3 or table 4 and comparing the functional expression with a reference sample.

### EXAMPLES

### Example I

Materials and Methods

Strains and Maintenance

General methods for culturing C. elegans strains were as described in Brenner (1974). Strains used in this study were: CB1500 (unc-93(e1500)), MT765 (unc-93(e1500 n224)), BC1958 (dpy-18(e364)/eT1 III; unc-46(e177)/eT1 V). A deletion mutant of msh-6: pk2504 was isolated from a chemical deletion library as described (Jansen et al., 1997).

Spontaneous Mutation Frequency

Growing cultures of msh-6 strains segregate a plethora of visible mutants indicative of a mutator phenotype. From the brood of four msh-6 hermaphrodites, 300 progeny animals were picked that had a wild-type appearance. These worms were grown individually and the progeny were inspected for mendelian segregation of visible phenotypes. Plates were screened a second time two days after food deprivation; this allows the scoring of an embryonic lethal phenotype, here interpreted as the abundant presence of dead eggs on the culture dish.

To determine whether msh-6 animals have a high incidence of the male (him) phenotype, broods of 3-5 animals of genotype msh-6 or wild-type were inspected for the presence of males. msh-6 animals yielded a him to hermaphrodite ratio of 1/1209 (0.08%), wild-type animals yielded a ration of 1/1059 (0.09%). The genetic recombination frequency was analyzed by determining the genetic distance between the visible marker unc-32 and dpy-18 on LGIII in an msh-6 and wild-type genetic background. For animals of genotype: msh-6; unc-32 dpy-18/++, the brood consisted of 412 wild-type, 20-Unc, 21-Dpy, and 112-Unc Dpy: resulting in a recombination frequency of 0.075 (Map distance: 7.5 cM). In a mismatch proficient genetic background the frequency was 0.072 (Map distance: 7.2 cM): 527 wild-type, 26-Unc, 24-Dpy, and 140-Unc Dpy segregated from animals of genotype unc-32 dpy-18/++.

The mutator phenotype of msh-6 C. elegans was quantified using the reciprocal translocation eT1 (III; V) as a balancer, as described by Rosenbluth (1983). First, msh-6 males were crossed with hermaphrodites that were homozygous for the translocated eT1 chromosomes (this genotype results in a visible phenotype because the translocation disrupts the unc-36 locus). F1 males were subsequently crossed with hermaphrodites of genotype: dpy-18; unc-46 (in order to mark the non-translocated chromosomes) and cross-progeny of the genotype msh-6/+I; dpy-18/eT1 III; unc-46/eT1 V were selected. Next generation animals, homozygous for msh-6 and segregating both Dpy-18 Unc-46 and Unc-36, were used as starting strains in the following experimental setup: Phenotypically wild-type progeny of hermaphrodites of the above-described genotype were picked onto individual plates and scored for segregation of the Dpy-18 Unc-46 phenotype. The frequency of recessive lethal mutations induced in the balanced area of the genome is reflected by the percentage of animals that fail to segregate this phenotype: a lethal in the crossover-suppressed region of the canonical chromosomes prevent embryos homozygous for these chromosomes from developing into adult Dpy Unc worms. Clonal lines that were positive in this screening were propagated and confirmed as carrying a lethal mutation inside one of the crossover-suppressed regions by showing no Dpy Unc phenotype in at least 250 offspring.

For determining the germline frequency in male sperm of msh-6 animals, males of genotype msh-6 I, dpy-18/eT1 III, unc-46/eT1 V were crossed to hermaphrodites of genotype eT1(III/V). Phenotypically, wild-type progeny were analyzed for segregation of the marked chromosomes as described above. The germline mutation frequency of hermaphrodite oocytes was determined by analyzing the phenotypically wild-type cross-progeny of dpy-18/eT1, unc-46/eT1 and eT1 males crossed to msh-6, dpy-18, unc-46 hermaphrodites. In the three crossing schemes, the msh-6-deficient animals that were used to start the analysis, were homozygous for more than one generation. Therefore, in order to prevent scoring mutations that occurred in earlier generations (which result in so-called “Jackpots”) more than 30 cross-progeny animals were tested from a single hermaphrodite.

RNA inhibition of msh-6 and msh-2 was performed by injecting hermaphrodites of strain BC1958 with cognate dsRNA and subsequent analysis of the mutator phenotype the progeny of the phenotypically wild-type F1. Thus, the F2 were inspected for segregation of the Dpy Unc phenotype. In addition, RNAi was measured by culturing BC1958 animals on msh-2 or msh-6 dsRNA-producing bacteria (described below).

Mutation Spectrum of msh-6 Worms

Phenotypic reversion of the uncoordinated “rubber-band,” egg-laying-defective phenotype conferred by unc-93(e1500) was used to determine the nature of mutations that occurred in a msh-6 genetic background. Cultures started with a single hermaphrodite of genotype msh-6 unc-93(e1500) were inspected regularly for revertants that were recognized by their wild-type movement and normal egg-laying behavior. Intragenic reversion events (mutations in at least four other loci can suppress the unc-93(e1500) associated phenotype) were identified by the failure of these alleles to complement unc-93(e1500n224). Subsequently, the coding region of the unc-93 locus was sequenced from animals that complimented unc-93(e1500n224).

Microsatellite Repeat Instability in msh-6 Worms

From a single hermaphrodite (msh-6 and Bristol N2), 55 progeny were picked to start lines that were maintained by transferring several L4 animals every 3-4 days to new plates. After ten generations, DNA was isolated from cultures started with a single animal (due to the mutator phenotype of msh-6, mutations will accumulate and often a sterile phenotype is observed when individual animals are cloned out). From these cultures, different genomic loci were analyzed by sequencing PCR products. Primers used are (5′-3′): R03C1_A: cggcaaacaatttttccg (SEQ ID NO: 1), R03C_C: acggaggtgttcacggag (SEQ ID NO:2), F59A3_A: cgtttgaaggatgatgtc (SEQ ID NO:3), F59A3_C: gatgctcgatgacttcgg (SEQ ID NO:4), C41D7_A: gattctcaagtccacccg (SEQ ID NO:5), C41D7_C: gacccgttctcctactcc SEQ ID NO:6), M03F4_A: cgaaatggatctgagtggg (SEQ ID NO:7), M03F4_C: atatcccatgatgacccc (SEQ ID NO:8), C24A3_A: gagtgcgcttgaagagactg (SEQ ID NO:9), C234A3_C: cggaactcggagagagatag (SEQ ID NO:10), Y54G11A_A: ggatcttggctcctggaacg (SEQ ID NO:11), Y54G11A_C: cattgagtgatactcggccg (SEQ ID NO:12).

Detection of Somatic Repeat Instability

To allow detection of somatic repeat instability, several constructs were created that contained stretches of either mono- or di-nucleotide repeats between the start of translation and the lacZ ORF, under the control of a heat-shock promoter.

In brief: vector L2681 (Fire-kit), that has a GFP/LacZ fusion under the control of a heat-shock promoter, was digested with BamHI and the vector relegated to create pRP1820; this cloning step removes two upstream ATG sequences without affecting essential promoter sequences. Then, the original starting codon was removed by site-directed mutagenesis to create pRP1821. This construct was subsequently used as a recipient for insertion of DNA fragments containing different types of repeats. Partially complementing oligonucleotides were annealed and inserted into a KpnI site near the beginning of the fusion protein encoded sequences. All constructs had a similar molecular architecture: Heat-shock promoter-(KpnI-)-ATG-(A or CA)n-GFP/LacZ ORF (sequences and cloning details available upon request). The different types of repeat used in this study were pRP1822: (A) 17, pRP1823: (A) 16, pRP1840: (A) 15, pRP1841: (CA) 15, pRP1842: (CA) 14, pRP1843 (CA) 13. pRP1823 and pRP1842 contain an in-frame LacZ construct encoding functional β-galactosidase.

All constructs were injected separately (together with pRF4 containing the dominant marker rol-6) into the canonical C. elegans strain BristolN2 to established transgenic lines (Mello et al., 1991). The transgenic array containing pRP1822 was integrated by y-irradiation and used for further detailed analysis of somatic reversion events.

To identify expression of β-galactosidase, nematodes were fixed and stained with X-gal (5-bromo-4-chloro-3-indolyl-β-D-galactpyranoside). Animals were examined with Nomarski optics.

cDNA Analysis

Primarily based on sequence homology comparison with other eukaryotic msh-6 genes, it was suspected that the GENEFINDER prediction of the C. elegans msh-6 coding sequence, Y47G6A.11, as annotated in the C. elegans database AceDB, was incorrect. While the N-terminal part of the predicted protein (encoded by Y47G6A.11 exon-1 to 7) does not show any significant homology with msh-6 orthologs, amino acids encoded by exon-8 are homologues to the N-terminal part of the human protein. This conclusion is favored by the fact that exon-8 predicts an ATG at +1 from a perfect SL1 splice site. SL1 splicing directly onto the putative exon-8, hereafter named exon-1 was confirmed by sequencing DNA material obtained from PCR on cDNA derived from Bristol N2 with primers corresponding to SL1 and msh-6 sequences. In addition, there was no ability to amplify cDNA with primers directed against the putative upstream exons and exon-8 or 9.

RNAi

By injection: PCR fragments of msh-6 and msh-2 coding sequences were cloned into vector pCCM114 (kind gift of Craig Mello) that contains oppositely oriented T7 promoters. Plasmid DNA was isolated, linearized and used as template to synthesize dsRNA in vitro with T7 RNA polymerase (Boehringer Mannheim) according to the manufacturer's conditions. Hermaphrodites were injected with 500 ng/μl dsRNA.

By feeding: msh-6 and msh-2 DNA segments were cloned into the “feeding vector”: L4440, and subsequently transformed to HT115 bacterial cells that were used for RNAi by feeding using the protocol described by Ahringer and coworkers (Fraser et al., 2000).

A library of bacterial clones, derived from the laboratory of Julie Ahringer (Welcome CRC, UK), that contains all C. elegans open reading frames was used to assay individual clones for their potential to induce replication errors, visualized by the detection of somatic repeat instability. To this end, individual animals that contain construct pRP1822 were placed on AGAR plates that were seeded with HT115 bacteria; each plate having a different bacterial clone and thus expressing RNA of a different C. elegans ORF. The next generation of C. elegans animals were assayed for expression of β-galactosidase activity, which is indicative of frame-shift errors that occurred in the transgene during development.

Screening of the Complete Genome of C. elegans:

Bacterial clones (HT110) that contain a plasmid, each plasmid carrying a specific DNA insert corresponding to a unique part of a C. elegans gene are seeded on standard assay plates as described in Fraser et al. (2000). The worms are grown for one or two generations, harvested, and assayed for LacZ expression as described above and in Tijsterman et al. (2002). If animals score “positive” for this assay (a significant level of expression is observed), the assay is repeated in 6-fold with the cognate bacterial clone. Bacterial clones that are validated by this method are considered to contain DNA sequence corresponding to genes that, when knocked down by RNA interference, lead to DNA instability. The genes corresponding to these DNA sequences are listed in tables 3 and 4. Because the bacterial clones are derived from a library of bacterial clones that were constructed for purposes as described here, the DNA sequence of the clones that are tested are known and kept in a database (see Fraser et al. (2000) for a detailed description of this library).

Results

Mutator Phenotype in Mismatch Repair Defective C. elegans

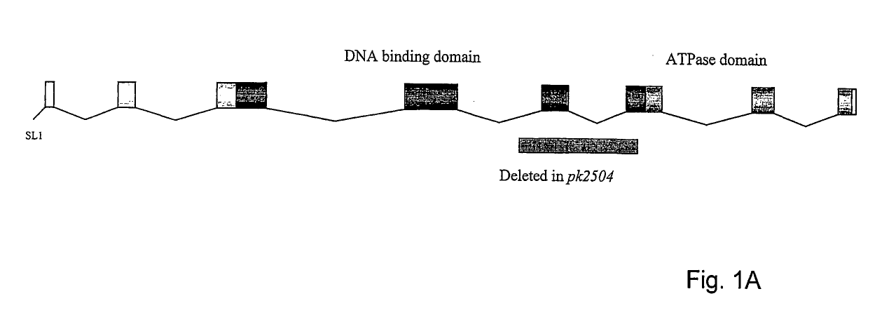

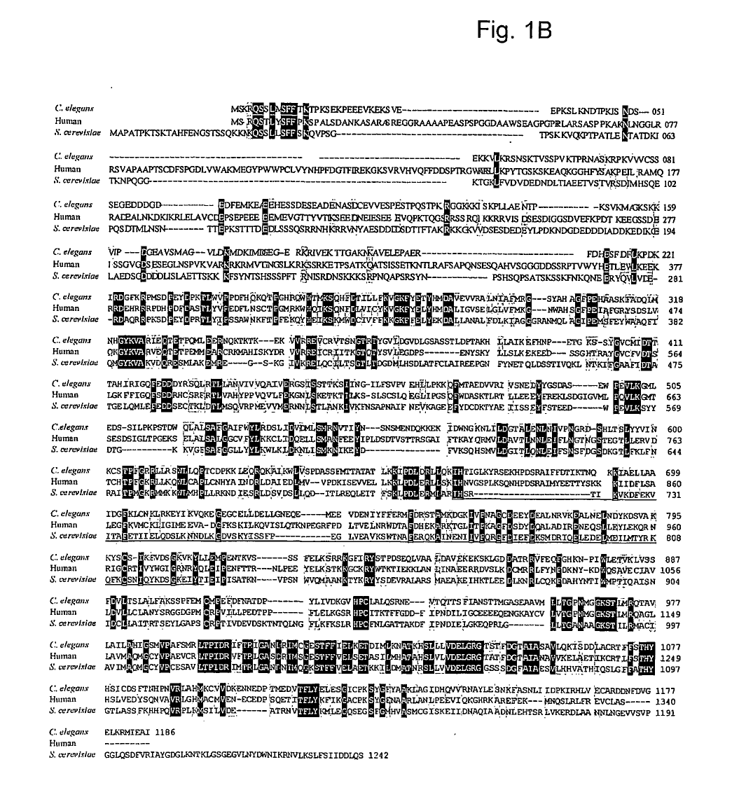

The genome sequence of C. elegans for homologues of bacterial and human DNA mismatch repair genes was screened, and msh-2 and msh-6 (homologues to the bacterial mutS gene) and mlh-1 and pms-1 (homologues to prokaryotic mutL) were found. Surprisingly, an orthologue of msh-3 was not detected. The msh-6 gene was then knocked out using the mutant library approach previously developed in the laboratory (Jansen et al., 1999). FIG. 1 shows the human and S. cerevisiae homologues aligned to msh-6 of C. elegans, and the deletion mutant that was used in this study.

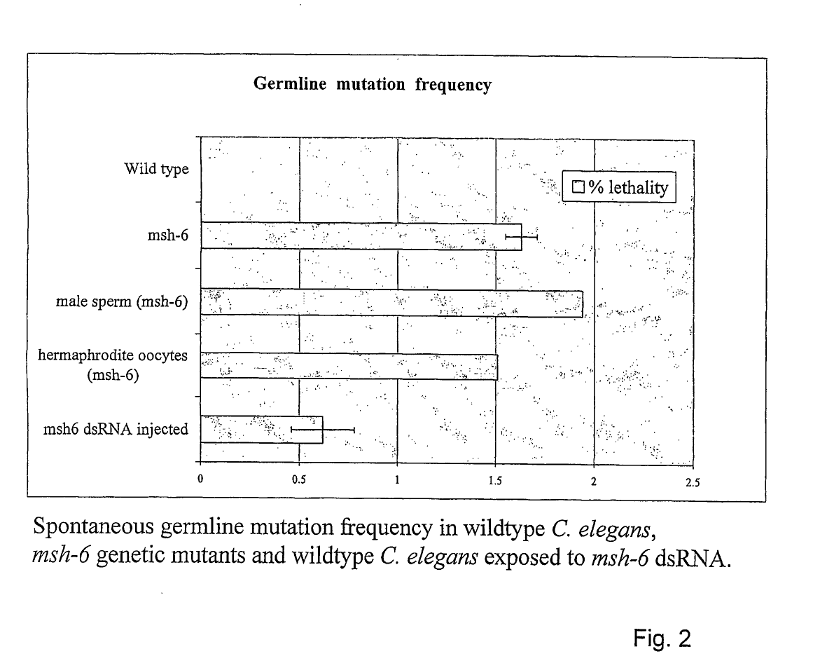

Homozygous msh-6 mutants are viable, and the first indication of the mutator phenotype was the frequent occurrence of readily recognizable mutants (Dpy, Unc) among the progeny. Since C. elegans lines can be maintained as self-fertilizing hermaphrodites, spontaneous new mutations can homozygose in self-progeny, so that recessive mutations are easily observed. (At least 20 phenotypic mutations were found in 300 progeny of two phenotypically wild-type hermaphrodites.) In the parental strain such level of spontaneous mutations is not seen. To quantify this mutator phenotype, lethal mutations were scored in a region of the genome that can be genetically monitored (see methods section). In a wild-type strain, spontaneous mutations were detected in this region below a frequency of 10⁻³, which is in line with the numbers reported in the literature (Rosenbluth et al., 1983). In msh-6 mutants, this level is at least 25-fold elevated (FIG. 2). Apart from the increased mutation frequency in the msh-6 mutant, no other phenotype that are indicative of specific defects in genome stability were noticed: X-chromosomal non-disjunction is not affected by the msh-6 deletion, indicated by the absence of a high incidence of male (him) phenotype. Also, no effect was observed on genetic recombination: the genetic distance between visible markers is similar in wild-type and msh-6 animals (see materials and methods for details).

These mutations could theoretically arise from mutations that occur uniquely in the sperm or in the oocytes of the hermaphroditic parent. To test whether the mismatch repair machinery protects the male as well as the female germline equally, experiments were performed that scored for spontaneous mutants in progeny from crosses between males and hermaphrodites, in which either one of the parents was mutant and the other wild-type for msh-6 (see methods for details). As shown in FIG. 2, both the oocytes of the hermaphroditic mother and the sperm from male fathers show a similar increase in the level of spontaneous mutagenesis in the msh-6 mutant. Two things were concluded: the frequency of original DNA replication errors is probably comparable in sperm and oocytes, and the level of protection by the mismatch repair machinery is also similar.

As a second measure of mutation rates, the frequency of loss-of-function mutations was taken in the unc-93(e1500) mutation. The e1500 allele makes animals hypercontracted, while complete loss of the unc-93 gene has no strong visible phenotype, and thus mutants of the unc-93(e1500) gene can be scored by recognizing normally moving animals among contracted ones. Therefore, this gene has been previously used to assay mutagenesis levels. It was found that the levels of mutations in unc-93(e1500) go up 30-fold in msh-6 mutants compared to wild-type.

The advantage of using the unc-93 monitor gene is that once obtained, these mutants can also be identified at the molecular level by direct sequencing of the relatively small genomic unc-93 gene. It is known that loss of four other genes (sup-9, sup-10, sup-11 and sup-18) also revert the unc-93(e1500) phenotype, so it was first sorted out the mutations that mapped to unc-93, and sequenced only those. The nature of the mutations is shown in table 1: found mostly were G to A transitions and frame-shifts in short monomeric runs, which is similar to the spectrum seen in bacteria, yeast and mammalian tissue culture cells. Note that nothing is known about point mutations in progeny of mismatch repair-deficient humans or animals, so that this is the first indication of spontaneous mutation spectra in progeny of repair-deficient animals.

Microsatellite instability is a hallmark of tumors derived from HNPCC patients. To see if and to what extent worms defective for msh-6 display microsatellite instability, 50 parallel lines were started by cloning the progeny of one msh-6 hermaphrodite. After these lines were maintained for ten generations, one animal per line was picked and sequenced various genomic loci-containing microsatellites. As shown in table 2, especially di-nucleotide repeats become highly instable in the absence of functional msh-6.

Having observed these fairly frequent repeat length changes in the germline of msh-6 mutants, the question of whether these changes could also be observed in somatic cells was determined. With worms living only two weeks, and most somatic cells being only a few cell divisions removed from the zygote, one may not expect too many mutations. Therefore, a sensitive system was devised for scoring repeat length instability.

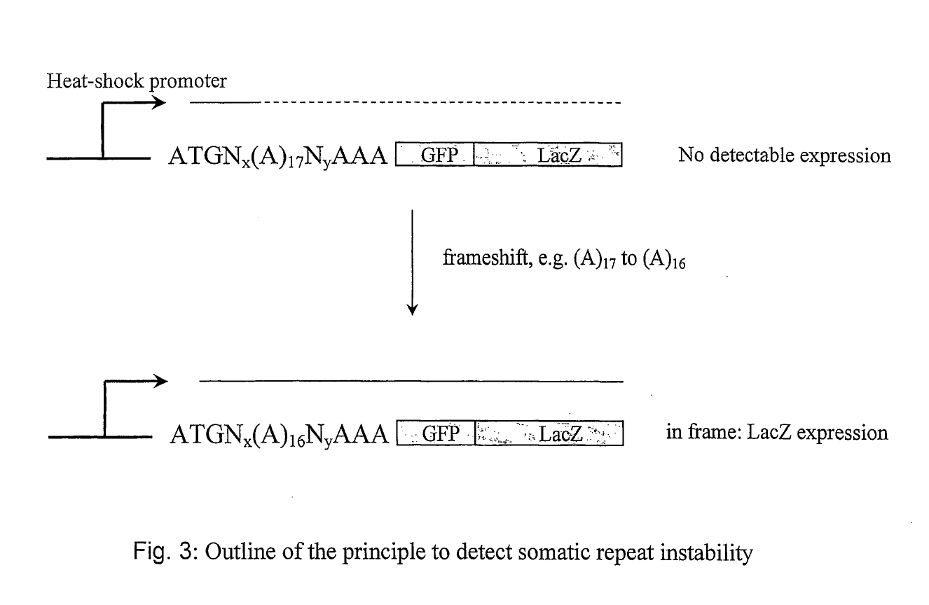

A repeat was cloned into a reporter gene, in such a way that the repeat was between the ATG initiation triplet and the domain of the gene encoding the enzymatic activity, which would keep the latter out-of-frame. Unrepaired replication errors in the repeat could bring the gene into the proper reading frame, which could be visualized (see also FIG. 3). To enhance the chances of finding such events, advantage was taken of the fact that transgenes in C. elegans are usually tandem repeats of hundreds of copies of the injected DNA. Therefore, a frame-shift in only one of those copies could be scored.

Initial attempts to use GFP for this purpose failed (presumably because the signal of one in-frame GFP gene copy among hundreds of out-of-frame copies was too low). A similar plasmid was then constructed, now using the LacZ reporter (FIG. 3). A disadvantage of this reporter is that the animal needs to be impregnated with the reagent X-gal, which kills the animal. An advantage is that LacZ staining can be more sensitive, especially because one can prolong the staining to get more signal.

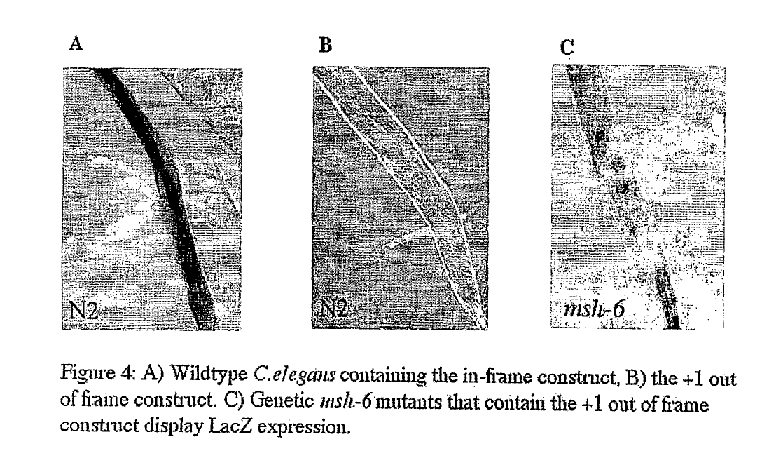

FIG. 4 shows staining of transgenic worms after the LacZ transgene is expressed by induction of the heat-shock promoter. In the wild-type worms, there is virtually no staining. The low level that is seen may reflect a low level of repeat instability even in the wild-type, or it may reflect frame-shift errors that are made during translation or both. In msh-6 mutant worms, on the other hand, the effect is dramatic, almost every worm shows one or more blue patches. It was concluded that these arise from repeat instability and restoration of the LacZ reading frame in lineages. Unfortunately, the fixated and stained worms have not allowed recognition of specific sublineages, but blue patches of multiple tissues were seen.

To check the role of the repeat in this msh-6-dependent frame-shift, transgenic animals were generated that contained identical constructs without the repeat and no animals were seen displaying the blue patched phenotype indicating that the repeat is an essential component of the detection system.

Destabilizing the Germline by Feeding msh-2 and msh-6 dsRNA

RNA interference is the silencing of gene expression by administration of dsRNA that corresponds to exonic sequences of that gene (Fire et al., 1998). The most striking effect is that dsRNA can be administered by soaking the worms in it (Tabara et al., 1999), or even by feeding them on E. coli that contain a plasmid that transcribes both strands which can hybridize to form dsRNA (Timmons and Fire). Worms were fed on E. coli that contained dsRNA for msh-6, and measured spontaneous mutation rates by scoring for mutants in the progeny. The results are shown in FIG. 1: the RNAi effect is comparable to that of a genetic knock-out of msh-6.

Destabilizing the Genetic Contents of Somatic Cells by Feeding msh-2 and msh-6 dsRNA

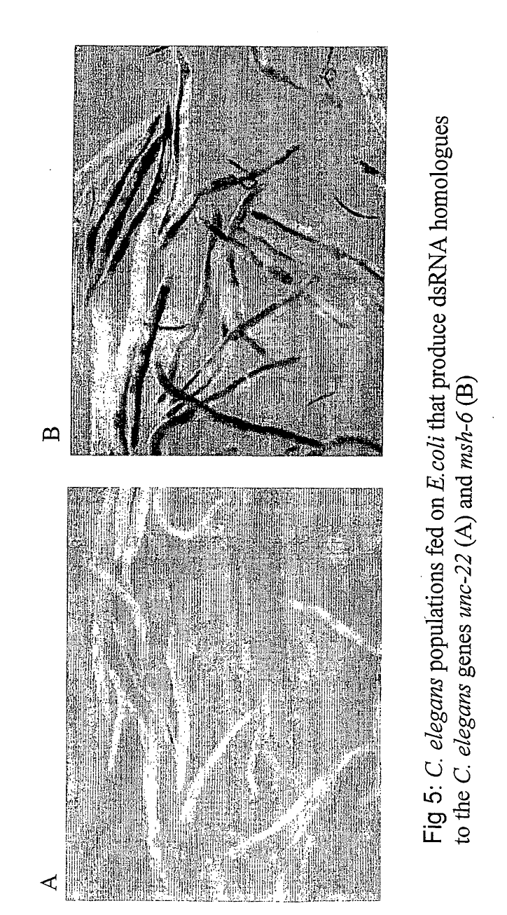

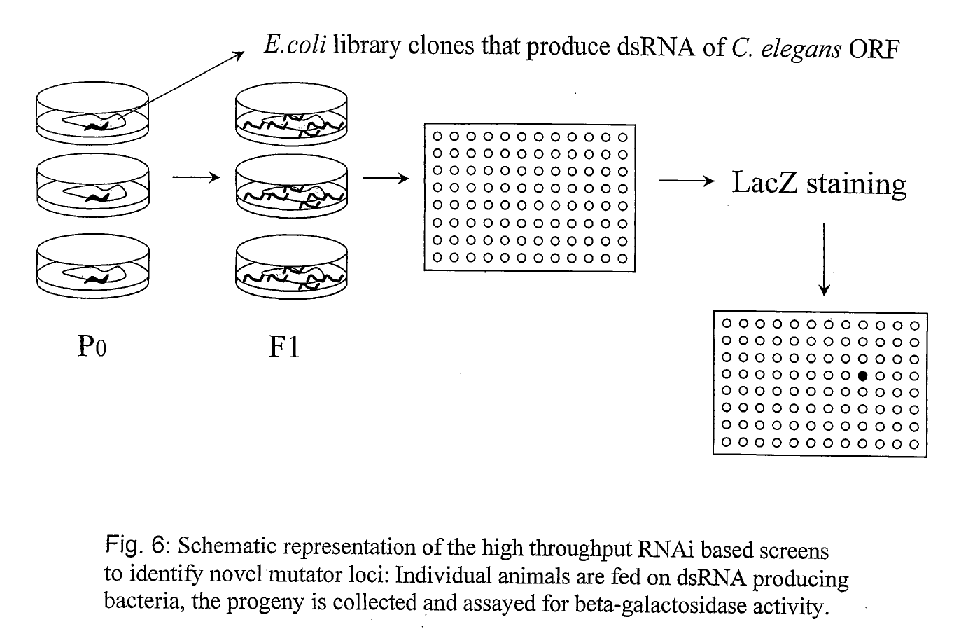

Combining the somatic repeat stability assay with msh-2 and msh-6 RNAi, dsRNA was fed to worms, and scored for repeat length changes in somatic cells. As shown in FIG. 5, the effect is the same as that of the genetic null: almost every animal has LacZ+ patches. This means that the stability of an animal's genome is directly influenced by the genetic material it eats.

TABLE 1

| Type of | | | |
|---|---|---|---|
| mutation | mutation | Position in unc-93 ORF. | a.a. change |
| Frameshift +1 | Insertion A | (221) TCGAGAA(A)TATTCGAA (235) | |
| +1 | Insertion A | (229) ATTCGAAAAA(A)CTTCG (243) | |
| +1 | Insertion A | (252) TTTGCAAAAA(A)TTTGG (266) | |
| +1 | Insertion A | (252) TTTGCAAAAA(A)TTTGG (266)* | |
| +1 | Insertion A | (372) TTCCAAAAAA(A)GAAG (285) | |
| −1 | Deletion T | (358) AAAGAGTTTTTCGAGG (373) | |
| Single basepair | G → A | (789) ATTTAACGGACTCCAA (804) | Gly → Arg |
| substitution. | G → A | (1155) ACACTGCGGACAAGTC (1170) | Gly → Arg |
| | G → A | (1551) TCTAGTTGGAGTTTAT (1566) | Gly → Arg |
| | G → A | (1650) TTCCCTAGTCTTCGGG (1665) | Val → Ile |
| | A → G | (1611) CTTTGTGATGGCCTGC (1626) | Met → Val |
| | A → C | (1492) AATATAAAGTTCATGT (1507) | Lys → Thr |
| | G → C | (1707) CGGAGCAGTAGTGAA (1721) | Val → Leu |
| | T → G | (1578) CGTCGGATGTGGCCTT (1593) | Cys → Gly |
| | T → G | GgctctgaggtttcagAAAAATGGCT (1443) | Disruption of 3′ |
| | | | splice site |
| Complex. | G → C +GC | (67) AAAAGTAG(GC)ATCACCG (81) | |
| | or | or | |
| | +C, G, +C | (68) AAAGTA(C)G(C)ATCACCG (81) | |
| | TTTTTG | (523) GATCATTTTTGCCCGA (538) | His → His |
| | ↓ | ↓ | and |
| | CTTTTT | (523) GATCACTTTTTCCCGA (538) | Cys → Phe |

Table 1. Unc-93(e1500) Mutation Spectrum in C. elegans msh-6:

The sequences correspond to SEQ ID NOS: 13-30, with the sequence marked by an * omitted, as repetitive, from the sequence listing.

TABLE 2

| | msh-6 | | | | | Wild-type | |
|---|---|---|---|---|---|---|---|
| Repeat | ^{C36C5}(A)₁₅ | ^{F59A3}(A)₁₅ | ^{R03C1}(A)₁₅ | ^{C41D7}(CA)₁₈ | ^{M03F4}(CA)₁₈ | ^{F59A3}(A)₁₅ | ^{M03F4}(CA)₁₈ |
| −1 | 0 | 3 | 2 | 7 | 5 | 0 | 0 |
| 0 | 44 | 42 | 38 | 32 | 34 | 44 | 44 |
| +1 | 0 | 0 | 0 | 2 | 6 | 0 | 0 |
| Total | 44 | 45 | 40 | 41 | 45 | 44 | 44 |

Table 2. Microsatellite Instability in the Genome of msh-6 Mutants

TABLE 3
List of found mutants

| Open reading frame | Similarity to known human genes |
|---|---|
| M04F3.1 | Replication Protein A subunit 2 (rpa-2) |
| B0511.8 | cdc-1 |
| D1081.8 | cdc-5 |
| F02E9.4 | sin-3 |
| R06C7.7 | |
| H26D21.2 | msh-2 |
| Y47G6A.11 | msh-6 |
| Y71F9AL.1/18 | 1: N6 adenine-specific DNA |
| | methyl(transfer)ase, N12 |
| | 18: Poly (ADP ribose) polymerase |
| F26E4.6 | cytochrome c oxidase subunit VIIc |
| C01A2.3 | cytochrome oxidase biogenesis protein like; OXA-1 |
| F22D6.4 | NADH ubiquinone oxidoreductase |
| | 13 kDa A subunit |
| F55A12.3 | PI-4P5′ kinase |
| E01A2.2 | arsenate resistance protein 2 ARS-2 |
| F25H2.9 | proteasome zeta chain |
| C36B1.4 | proteasome A type subunit |
| F39H11.5 | proteasome beta chain |

TABLE 4

| Gene name | Accession No. | Similarity to known human genes |
|---|---|---|
| M04F3.1 | NM_059045 | Rpa-2 |
| B0511.8 | NM_060382 | Cdc-1 |
| D1081.8 | NM_059902 | Cdc-5 |
| F02E9.4 | NM_059883 | Sin-3 |
| R06C7.7 | NM_059649 | |
| H26D21.2 | AF106587 | Msh-2 |
| Y47G6A.11 | AC024791 | Msh-6 |
| Y71F9AL.18 | NM_058671 | Poly(adp ribose) polymerase |
| F55A12.3 | AF003130 | PI-4P5′ kinase |
| E01A2.2 | NM_058901 | Arsenate resistance protein 2 (ars2) |
| F26E4.6 | NM_060195 | Cytochrome c oxidase su. VIIc |
| C01A2.3 | NM_060955 | Oxa 1 |
| F22D6.4 | NM_059606 | NADH ubiquinone oxidoreductase |
| | | 13 kDa su. |
| F25H2.9 | NM_060364 | Proteasome Z chain |
| C36B1.4 | NM_059959 | Proteasome A type su. |
| F39H11.5 | Z81079 | Proteasome beta chain |
| T02H6.11 | NM_061394 | Ubiquinol cyt. C reductase complex su. |
| F54D10.1 | AF099917 | Skr-15 SKP1 like |
| K07D4.3 | AF077534 | Rpn-11 |
| C17G10.4 | U28739 | Cdc-14 |
| C25H3.3 | NM_062714 | |
| C25H3.4 | NM_062713 | Translation initiation factor SUI1 |
| C32D5.6 | NM_062872 | |
| T19D12.5 | NM_062948 | Casein kinase I |
| B0495.2 | NM_063216 | Cdc-2 |
| F49E12.6 | NM_063370 | RBB-3 like |
| T10B9.5 | NM_063709 | Cytochrome P450 |
| R06F6.8a | Z46794 | |
| R03D7.2 | NM_063953 | |
| F32A11.2 | Z81521 | Hpr-17/rad-17 |
| B0412.3 | NM_064863 | |
| R74.4 | NM_065438 | Heat-shock protein |
| F20H11.5 | NM_066052 | D-amino acid oxidase |
| T26A5.5 | U00043 | |
| B0361.1 | | Cwf-19 |
| H14A12.3 | NM_066240 | |
| T23G5.6 | NM_066641 | TdT interacting protein |
| Y56A3A.29 | AL132860 | Uracil-DNA glycosylase |
| T28D6.4 | NM_067060 | |
| Y111B2A.1 | NM_067230 | AFC2 like/CLK2-4 like |
| Y76A2B.5 | NM_067400 | |
| Y43F4B.1 | NM_067336 | |
| ZK520.3 | NM_067423 | |
| Y56A3A.33 | NM_067164 | Exonuclease similarity to antigen GOR |
| Y39A3CL.4a | AC024763 | |
| Y62E10A.6 | NM_070172 | NADPH: adrenodoxin oxidoreductase |
| F29C4.6 | NM_067464 | |
| AC8.1 | NM_075638 | Poly (adp-ribose) polymerase |
| F15E6.1 | NM_068138 | |
| K08D10.2 | NM_068105 | |
| T05A12.4 | NM_068659 | |
| C33D9.5 | NM_069115 | Rad-50 like |
| K08F4.1 | NM_069440 | |
| K08E7.7 | NM_070011 | Cullin cul-6 |
| K09B11.2 | NM_070187 | |
| F14F9.5 | NM_071972 | AP-endonuclease like |
| F44C4.4 | NM_072280 | Lin-15b like |
| ZC196.6 | NM_072846 | |
| ZK856.1 | NM_073215 | Cul-5 cullin |
| C06H2.3 | NM_073430 | |
| F08H9.4 | NM_074185 | Heat-shock protein hsp20 |
| F43D2.1 | NM_074214 | Cyclin C G1/S like |
| C30G7.1 | NM_074279 | Histone H1 like |
| C25D7.6 | Z81079 | MCM-3 |
| F28E4.1 | | Cytochrome P450 |
| Y113G7A.9 | NM_075475 | |
| W07A8.3 | NM_075601 | |
| F57C12.2 | NM_075717 | |
| F19G12.2 | NM_075868 | Ribonucleotide reductase |
| R07E4.2 | NM_076596 | SPT associated factor 42 like |
| C09B8.6 | NM_076608 | Heat-shock protein hsp20 |
| F45E1.6 | NM_076943 | Histone H3 |
| C44C10.2 | NM_077558 | Cytochrome P450 |
| F46G10.3 | NM_077819 | SIR2 family of genes |
| F02D10.7 | NM_077840 | |
| C53A5.3 | Z81486 | Hdac1 |
| C35A5.9 | NM_073298 | Hdac2 |
| H12C20.2a | AL022272 | Pms-2 |
| T28A8.7 | Z92813 | Mlh-1 |

### REFERENCES

Aaltonen et al. (1993) Science 260: 812-816

Baker et al. (1996) Nat. Genet. 13: 336-342

Brenner (1974) Genetics 77: 71-94

Bronner et al. (1994) Nature 368: 258-261

Brummelkamp et al. (2002) Sciencexpress 21 Mar. 2002, 1-6

Cunningham et al. (1998) Cancer Res. 58: 3455-3460

de Wind et al. (1995) Cell 82: 321-330

Edelman et al. (1996) Cell 85: 1125-1134

Elbashir et al. (2002) Nature 411:494-498

Fishel et al. (1993) Cell 75: 1027-1038

Fire et al. (1998) Nature 391: 806-811

Fraser et al. (2000) Nature 408: 325-330

Herman et al. (1998) Proc. Natl. Acad. Sci. USA 95: 6870-6875

Ionov et al. (1993) Nature 260: 558-561

Jansen et al. (1999) Nat. Genet. 17: 119-121

Kane et al. (1997) Cancer Res. 57: 808-811

Kolodner et al. (1994) Cold Spring Harb. Symp. Quant. Biol. 59: 331-338

Leach et al. (1993) Cell 75: 1215-1225

Liu et al. (1996) Nat. Med. 2: 169-174

Loeb (1991) Cancer Res. 51: 3075-3079

Lynch et al. (1998) Oncology 55: 103-108

Mello et al. (1991) EMBO. J. 10: 3959-3970

Narayanan et al. (1997) Proc. Natl. Acad. Sci. USA 94: 3122-3127

Nicolaides et al. (1994) Nature 371: 75-80

Papadopoulos et al. (1994) Science 268: 1915-1917

Peinado et al. (1992) Proc. Natl. Acad. Sci. USA 89: 10065-10069

Peltomaki et al. (1993) Cancer Res. 53: 5853-5855

Peltomaki and de la Chapelle (1997) Adv. Cancer Res. 71: 93-119

Prolla et al. (1998) Nat. Genet. 18: 276-279

Reitmar et al. (1995) Nat. Genet. 11: 64-70

Rosenbluth et al. (1983) Mut. Res. 110: 39-48

Tabara et al. (1999) Science 282: 430-431

Tijsterman, M., Pothof, J., and Plasterk, R. H. A. (2002). Frequent germline mutations and somatic repeat instability in DNA mismatch repair instability in DNA mismatch repair-deficient C. elegans. Submitted.

Timmons and Fire (1999) Nature 395: 854

Veigl et al. (1998) Proc. Natl. Acad. Sci. USA 95: 8698-8702

What is claimed is:

1. A method for determining whether a gene product is involved in preventing a replication error in a cell, the method comprising:
providing a cell with a specific inhibitor for a gene product; and
determining the expression level of a marker gene in the cell, wherein the expression level of the marker gene is dependent on the occurrence of a replication error.

2. The method according to claim 1, wherein the replication error comprises a nucleic acid repeat instability.

3. The method according to claim 1, wherein the specific inhibitor for a gene product comprises using gene-specific RNAi.

4. The method according to claim 3, wherein the specific inhibitor for a gene product comprises gene-specific double-stranded RNAi.

5. The method according to claim 1, wherein the cell is present in a non-human organism.

6. The method according to claim 5, wherein said organism comprises C. elegans.

7. The method according to claim 1, further comprising providing the marker gene to the cell.

8. The method according to claim 7, wherein the marker gene comprises LacZ.

9. The method according to claim 1, wherein the expression level of the marker gene is dependent on a nucleic acid repeat in the marker gene.

10. The method according to claim 9, wherein the repeat, or an incorrect repair of the repeat, results in a frame-shift within the coding region of the marker gene.

11. The method according to claim 10, wherein the frame-shift results in a functional protein.

12. The method according to claim 11, wherein an activity of the functional protein is detected.

13. The method according to claim 12, wherein the activity comprises β-galactosidase activity.

14. The method according to claim 1, further comprising identifying a gene involved in preventing nucleic acid repeat instability in the cell.

15. An isolated and/or recombinant gene produced by the process comprising
providing a cell with a specific inhibitor for a gene product; and
determining the expression level of a marker gene in the cell, wherein the expression level of the marker gene is dependent on the occurrence of a replication error; and
identifying a gene involved in preventing nucleic acid repeat instability in the cell.

16. The isolated and/or recombinant gene according to claim 15, wherein the gene is selected from the group consisting of at least one of Y71F9AL.1/18, F26E4.6, C01A2.3, F22D6.4, F55A12.3, E01A2.2, F25H2.9, C36B1.4, F39H11.5, M04F3.1, B0511.8, D1081.8, F02E9.4, R06C7.7, H26D21.2, Y47G6A.11, Y71F9AL.18, F55A12.3, E01A2.2, F26E4.6, C01A2.3, F22D6.4, F25H2.9, C36B1.4, F39H11.5, T02H6.11, F54D10.1, K07D4.3, C17G10.4, C25H3.3, C25H3.4, C32D5.6, T19D12.5, B0495.2, F49E12.6, T10B9.5, R06F6.8a, R03D7.2, F32A11.2, B0412.3, R74.4, F20H11.5, T26A5.5, B0361.1, H14A12.3, T23G5.6, Y56A3A.29, T28D6.4, Y111B2A.1, Y76A2B.5, Y43F4B.1, ZK520.3, Y56A3A.33, Y39A3CL.4a, Y62E10A.6, F29C4.6, AC8.1, F15E6.1, K08D10.2, T05A12.4, C33D9.5, K08F4.1, K08E7.7, K09B11.2, F14F9.5, F44C4.4, ZC196.6, ZK856.1, C06H2.3, F08H9.4, F43D2.1, C30G7.1, C25D7.6, F28E4.1, Y113G7A.9, W07A8.3, F57C12.2, F19G12.2, R07E4.2, C09B8.6, F45E1.6, C44C10.2, F46G10.3, F02D10.7, C53A5.3, C35A5.9, H12C20.2a, and T28A8.7.

17. A method for determining whether a cell is predisposed to display a nucleic acid repeat instability phenotype, the method comprising:
determining functional expression of a gene according to claim 15, or an equivalent or homologue thereof, in a cell.

18. A method according to claim 17, wherein the cell is in a non-human organism.

19. A method according to claim 17, wherein the cell is present in a clinical sample.

20. A method according to claim 19, further comprising determining whether an individual is predisposed to display a nucleic acid repeat instability phenotype.

21. A method according to claim 19, further comprising determining whether the cell is a cancer cell.

22. A kit for determining whether a cell is predisposed to display a nucleic acid repeat instability phenotype, comprising a means for determining functional expression of a gene selected from the group consisting of at least one of Y71F9AL.1/18, F26E4.6, C01A2.3, F22D6.4, F55A12.3, E01A2.2, F25H2.9, C36B1.4, F39H11.5, M04F3.1, B0511.8, D1081.8, F02E9.4, R06C7.7, H26D21.2, Y47G6A.11, Y71F9AL.18, F55A12.3, E01A2.2, F26E4.6, C01A2.3, F22D6.4, F25H2.9, C36B1.4, F39H11.5, T02H6.11, F54D10.1, K07D4.3, C17G10.4, C25H3.3, C25H3.4, C32D5.6, T19D12.5, B0495.2, F49E12.6, T10B9.5, R06F6.8a, R03D7.2, F32A11.2, B0412.3, R74.4, F20H11.5, T26A5.5, B0361.1, H14A12.3, T23G5.6, Y56A3A.29, T28D6.4, Y111B2A.1, Y76A2B.5, Y43F4B.1, ZK520.3, Y56A3A.33, Y39A3CL.4a, Y62E10A.6, F29C4.6, AC8.1, F15E6.1, K08D10.2, T05A12.4, C33D9.5, K08F4.1, K08E7.7, K09B11.2, F14F9.5, F44C4.4, ZC196.6, ZK856.1, CO₆H2.3, F08H9.4, F43D2.1, C30G7.1, C25D7.6, F28E4.1, Y113G7A.9, W07A8.3, F57C12.2, F19G12.2, R07E4.2, C09B8.6, F45E1.6, C44C10.2, F46G10.3, F02D10.7, C53A5.3, C35A5.9, H12C20.2a, T28A8.7 and an equivalent or homologue thereof.

23. A kit according to claim 22, comprising an antibody specific for a gene product of the gene.

24. A kit according to claim 22, comprising a probe for the gene.

25. A kit according to claim 22, comprising a means for obtaining a sequence of the gene.

26. A method for determining whether a compound is capable of influencing a process involved in preventing a replication error in a cell, the method comprising:
providing a cell with a compound; and
determining the expression level of a marker gene in the cell, wherein the expression level of the marker gene is dependent on a replication error.

27. A method according to claim 26, further comprising providing the cell with a specific inhibitor for the expression of a gene involved in preventing a replication error in the cell.

28. A method according to claim 27, wherein the gene involved in preventing a replication error in the cell, is a gene selected from the group consisting of at least one of Y71F9AL.1/18, F26E4.6, C01A2.3, F22D6.4, F55A12.3, E01A2.2, F25H2.9, C36B1.4, F39H11.5, M04F3.1, B0511.8, D1081.8, F02E9.4, R06C7.7, H26D21.2, Y47G6A.11, Y71F9AL.18, F55A12.3, E01A2.2, F26E4.6, C01A2.3, F22D6.4, F25H2.9, C36B1.4, F39H11.5, T02H6.11, F54D10.1, K07D4.3, C17G10.4, C25H3.3, C25H3.4, C32D5.6, T19D12.5, B0495.2, F49E12.6, T10B9.5, R06F6.8a, R03D7.2, F32A11.2, B0412.3, R74.4, F20H11.5, T26A5.5, B0361.1, H14A12.3, T23G5.6, Y56A3A.29, T28D6.4, Y11B2A.1, Y76A2B.5, Y43F4B.1, ZK520.3, Y56A3A.33, Y39A3CL.4a, Y62E10A.6, F29C4.6, AC8.1, F15E6.1, K08D10.2, T05A12.4, C33D9.5, K08F4.1, K08E7.7, K09B11.2, F14F9.5, F44C4.4, ZC196.6, ZK856.1, C06H2.3, F08H9.4, F43D2.1, C30G7.1, C25D7.6, F28E4.1, Y113G7A.9, W07A8.3, F57C12.2, F19G12.2, R07E4.2, C09B8.6, F45E1.6, C44C10.2, F46G10.3, F02D10.7, C53A5.3, C35A5.9, H12C20.2a, T28A8.7 and an equivalent or homologue thereof.

29. A gene delivery vehicle comprising a nucleic acid according to claim 15.

30. A method for influencing a process involved in preventing a replication error in a cell comprising providing the cell with a gene delivery vehicle according to claim 29.

31. A method of treating a subject, comprising administering to a subject the a gene delivery vehicle according to claim 29.

32. The non-human animal comprising a marker gene wherein the level of expression of said marker gene is dependent on the occurrence of said replication error.

33. The non-human animal according to claim 32 wherein the marker gene is provided to cells of the animal.

34. The non-human animal according to claim 33, wherein the animal is transgenic for the marker gene.

35. The method according to claim 26, further comprising providing a non-human animal having the marker gene and determining in the animal or progeny thereof whether the expression level of the marker gene is altered.

36. The method according to claim 35, wherein the non-human animal comprises C. elegans.

37. The method according to claim 36, wherein said compound comprises RNAi, or a free radical.

38. The method according to claim 37, wherein said RNAi is specific for a gene selected from the group consisting of at least one of Y71F9AL.1/18, F26E4.6, C01A2.3, F22D6.4, F55A12.3, E01A2.2, F25H2.9, C36B1.4, F39H11.5, M04F3.1, B0511.8, D1081.8, F02E9.4, R06C7.7, H26D21.2, Y47G6A.11, Y71F9AL.18, F55A12.3, E01A2.2, F26E4.6, C01A2.3, F22D6.4, F25H2.9, C36B1.4, F39H11.5, T02H6.11, F54D10.1, K07D4.3, C17G10.4, C25H3.3, C25H3.4, C32D5.6, T19D12.5, B0495.2, F49E12.6, T10B9.5, R06F6.8a, R03D7.2, F32A11.2, B0412.3, R74.4, F20H11.5, T26A5.5, B0361.1, H14A12.3, T23G5.6, Y56A3A.29, T28D6.4, Y111B2A.1, Y76A2B.5, Y43F4B.1, ZK520.3, Y56A3A.33, Y39A3CL.4a, Y62E10A.6, F29C4.6, AC8.1, F15E6.1, K08D10.2, T05A12.4, C33D9.5, K08F4.1, K08E7.7, K09B11.2, F14F9.5, F44C4.4, ZC196.6, ZK856.1, CO₆H2.3, F08H9.4, F43D2.1, C30G7.1, C25D7.6, F28E4.1, Y113G7A.9, W07A8.3, F57C12.2, F19G12.2, R07E4.2, C09B8.6, F45E1.6, C44C10.2, F46G10.3, F02D10.7, C53A5.3, C35A5.9, H12C20.2a, and T28A8.7.

39. A method for typing a cell, the method comprising:
determining, in a sample having a cell, the functional expression of a gene selected from the group consisting of at least one of Y71F9AL.1/18, F26E4.6, C01A2.3, F22D6.4, F55A12.3, E01A2.2, F25H2.9, C36B1.4, F39H11.5, M04F3.1, B0511.8, D1081.8, F02E9.4, R06C7.7, H26D21.2, Y47G6A.11, Y71F9AL.18, F55A12.3, E01A2.2, F26E4.6, C01A2.3, F22D6.4, F25H2.9, C36B1.4, F39H11.5, T02H6.11, F54D10.1, K07D4.3, C17G10.4, C25H3.3, C25H3.4, C32D5.6, T19D12.5, B0495.2, F49E12.6, T10B9.5, R06F6.8a, R03D7.2, F32A11.2, B0412.3, R74.4, F20H11.5, T26A5.5, B0361.1, H14A12.3, T23G5.6, Y56A3A.29, T28D6.4, Y11B2A.1, Y76A2B.5, Y43F4B.1, ZK520.3, Y56A3A.33, Y39A3CL.4a, Y62E10A.6, F29C4.6, AC8.1, F15E6.1, K08D10.2, T05A12.4, C33D9.5, K08F4.1, K08E7.7, K09B11.2, F14F9.5, F44C4.4, ZC196.6, ZK856.1, C06H2.3, F08H9.4, F43D2.1, C30G7.1, C25D7.6, F28E4.1, Y113G7A.9, W07A8.3, F57C12.2, F19G12.2, R07E4.2, C09B8.6, F45E1.6, C44C10.2, F46G10.3, F02D10.7, C53A5.3, C35A5.9, H12C20.2a, T28A8.7 and an equivalent or homologue thereof; and
comparing the functional expression of the gene with a reference sample.
